(19) United States
(12) Patent Application Publication
(10) Pub. No.: US 2007/0287826 A1
(43) Pub. Date: Dec. 13, 2007

(54) PRODUCTION OF HIGH MOLECULAR MASS LECTINS

(76) Inventor: FINN MATTHIESEN, Bronshoj (DK)

Correspondence Address: BROWDY AND NEIMARK, P.L.L.C.;624 NINTH STREET, NW, SUITE 300, WASHINGTON, DC, 20001-5303, US

(21) Appl. No.: 11/756,037

(22) Filed: May 31, 2007

Related U.S. Application Data
(63) Continuation of application No. 10/484,501, filed on Jan. 22, 2004, now abandoned.

(30) Foreign Application Priority Data
Jul. 23, 2001 (DK) PA200101139

(51) Int. Cl. C07K 14/42

(52) U.S. Cl. 530/396

(57) ABSTRACT

The invention relates to a process for preparing a high molecular weight lectin composition, in particular comprising the mannose-binding lectin (MBL). In one aspect there is provided a method for producing a composition comprising a variety of lectin molecules, wherein substantially all of said lectin molecules have a high molecular weight (above the molecular weight for dimer lectins). A precipitating agent is added to a lectin preparation comprising lectin molecules having a high molecular weight and lectin molecules having a low molecular weight (below or equal to the molecular weight for dimer lectins), and a precipitate and a supernatant are allowed to form. By separating said precipitate from said supernatant, a precipitate fraction comprising the high molecular weight lectin molecules is obtained.

The invention relates to a process for preparing a high molecular weight lectin composition, in particular comprising the mannose-binding lectin (MBL), suitable for using recombinantly produced lectins as starting material, a high molecular weight lectin composition, a pharmaceutical composition comprising same, as well as the use of the produced composition for the preparation of a pharmaceutical composition for treating various conditions and diseases.

### BACKGROUND OF THE INVENTION

Mannose-binding protein (MBL) is a protein to be used for substitution or replacement therapy in patients with inherited or acquired MBL-deficiency associated with functional and/or clinical symptoms.

MBL is a protein of the collectin family and characterized by an oligomeric structure of subunits each consisting of a calcium-dependent, C-type carbohydrate-recognition domain (CRD), attached to a collagenous rod. MBL activates the complement system via associated serine proteases (MASP—mannose-binding lectin associated serine proteases), i.e. by a mechanism similar to C1q.

MBL derived from human blood plasma is assembled into an oligomer of subunits, each consisting of three identical polypeptide chains. The number of subunits in an MBL molecule varies [Lipscombe R J, et al., Distinct physicochemical characteristics of human mannose binding protein expressed by individuals of differing genotype, Immunology 85 (1995) 660-667.], but it has been suggested that the biologically active polypeptide is an oligomer consisting of more than three subunits. Plasma comprises oligomers of more than three subunits as well as denatured and structurally impaired protein forms leading to bands on for example SDS gels between the dominating MBL bands corresponding to the higher oligomers.

Recombinantly produced MBL reveals an oligomer variation similar to plasma-derived MBL [Vorup-Jensen T et al: Recombinant expression of human mannan-binding lectin, Int Immunopharm 1 (2001) 677-687]. However, usually recombinantly produced MBL has a higher content of low-mass forms than do plasma derived MBL. Low-mass forms of MBL include for example single polypeptide chains, single subunits, and dimeric subunits.

In PCT application WO00/70043 a method for separating high oligomers from low mass forms is described, wherein recombinantly produced MBL is subjected to fractionation on a special column.

Precipitation of protein-containing fractions have been described in the art. For example in Storgaard P, Nielsen E H, Andersen O, Skriver E, Mortensen H, Hojrup P, Leslie G, Holmskow U, Svehag S E. Isolation and characterization of porcine mannan-binding proteins of different size and ultrastructure. Scand J Immunol 1996; 43:289-96 isolation of MBL from porcine serum is reported, wherein MBPs were purified by use of PEG precipitation, affinity chromatography on mannan-Sepharose, protein A- and anti-porcine IgM-Sepharose followed by gel filtration.

In WO99/64453 a Cohn fractionation of plasma through the use of ethanol precipitation was described. The obtained fraction comprised among other molecules MBL.

### DESCRIPTION OF THE INVENTION

A fast and simple method to isolate lectins, such as mannose-binding lectins (MBL), or derivatives and variants thereof (herein collectively designated lectins) from a solution is of major importance.

Moreover, to be in control of the mass distribution of lectins is also of major importance, because different oligomers of said lectin polypeptides possess different biological functions and activity.

The present invention describes a method to isolate lectins, such as MBL, by precipitation by adding a precipitating agent to the solution. The method can be used as a unit operation during preparation, purification, and/or formulation of said polypeptide. Compared to other methods, it is advantageous that the method of invention can be applied to change the composition of oligomers of said polypeptide, so that high-mass oligomers of said polypeptide are separated from low-mass oligomers of said polypeptide.

Thus, in one aspect the invention relates to a method for changing the oligomer distribution of lectins in solution. In one embodiment the invention relates to a method for increasing the ratio R of a composition comprising a variety of lectin molecules, wherein R is the ratio of the concentration of lectin molecules having a high molecular weight above the molecular weight for dimer lectins to the concentration of lectin molecules having a low molecular weight below or equal to the molecular weight for dimer lectins, said method comprising,
obtaining a lectin preparation comprising low molecular weight lectin and high molecular weight lectin, said preparation having the ratio R=R₀,
adding to said preparation a precipitating agent and allowing a precipitate and a supernatant to form,
separating said precipitate from said supernatant, obtaining a precipitate fraction having the ratio R=R₁ wherein R₁>R₀, and optionally obtaining a supernatant fraction having the ratio R=R₂, wherein R₂<R₀,
optionally resuspending said precipitate fraction,
obtaining a composition comprising the lectin molecules of the precipitate fraction.

Thereby a composition having an increased content of high mass oligomers of lectins as compared to the starting material is obtained.

In particular the increase of the ratio leads to compositions comprising lectins having a molecular weight above the molecular weight for dimer lectins. Thus, in another aspect the invention relates to a method for producing a composition comprising a variety of lectin molecules, wherein substantially all of said lectin molecules have a high molecular weight above the molecular weight for dimer lectins, said method comprising,
obtaining a lectin preparation comprising lectin molecules having a high molecular weight above the molecular weight for dimer lectins and lectin molecules having a low molecular weight below or equal to the molecular weight for dimer lectins, said preparation having the ratio R=R₀, wherein R is the ratio of the concentration of lectin molecules having a high molecular weight above the molecular weight for dimer lectins to the concentration of lectin molecules having a low molecular weight below or equal to the molecular weight for dimer lectins,
adding to said preparation a precipitating agent and allowing a precipitate and a supernatant to form,
separating said precipitate from said supernatant, obtaining a precipitate fraction having the ratio R=R₁ wherein R₁>R₀, and optionally obtaining a supernatant fraction having the ratio R=R₂, wherein R₂<R₀,
optionally resuspending said precipitate fraction,
obtaining a composition comprising the lectin molecules of the precipitate fraction.

Since the starting material for the methods comprises high mass lectins as well as low mass lectins, the invention also relates to a method for separating a composition comprising a variety of lectin molecules, wherein substantially all of said lectin molecules have a high molecular weight above the molecular weight for dimer lectins, from a lectin preparation comprising lectin molecules having a high molecular weight above the molecular weight for dimer lectins and lectin molecules having a low molecular weight below or equal to the molecular weight for dimer lectins, said preparation having the ratio R=R₀, wherein R is the ratio of the concentration of lectin molecules having a high molecular weight above the molecular weight for dimer lectins to the concentration of lectin molecules having a low molecular weight below or equal to the molecular weight for dimer lectins, said method comprising,
obtaining said lectin preparation,
adding to said preparation a precipitating agent and allowing a precipitate and a supernatant to form,
separating said precipitate from said supernatant, obtaining a precipitate fraction having the ratio R=R₁ wherein R₁>R₀, and optionally obtaining a supernatant fraction having the ratio R=R₂, wherein R₂<R₀,
optionally resuspending said precipitate fraction,
obtaining a composition comprising the lectin molecules of the precipitate fraction.

For most purposes the high mass lectins are the desired, however, for some applications the low mass lectins are wanted, and accordingly, the invention further relates to a method for producing a composition comprising a variety of lectin molecules, wherein substantially all of said lectin molecules have a high molecular weight below or equal to the molecular weight for dimer lectins, said method comprising,
obtaining a lectin preparation comprising lectin molecules having a high molecular weight above the molecular weight for dimer lectins and lectin molecules having a low molecular weight below or equal to the molecular weight for dimer lectins, said preparation having the ratio R=R₀, wherein R is the ratio of the concentration of lectin molecules having a high molecular weight above the molecular weight for dimer lectins to the concentration of lectin molecules having a low molecular weight below or equal to the molecular weight for dimer lectins,
adding to said preparation a precipitating agent and allowing a precipitate and a supernatant to form,
separating said precipitate from said supernatant, obtaining a supernatant fraction having the ratio R=R₂, wherein R₂<R₀, and optionally obtaining a precipitate fraction having the ratio R—R₁ wherein R₁>R₀,
obtaining a composition comprising the lectin molecules of the supernatant fraction.

For all the methods mentioned above, preferably the precipitate fraction has the ratio R=R₁, wherein R₁ is at least 1, such as at least 1.05, such as at least 1.10, such as at least 1.15, such as at least 1.25, such as at least 1.50, such as at least 1.75, such as at least 2.0, such as at least 2.5, such as at least 3.0, such as at least 4.0, such as at least 5.0, such as at least 6.0, such as at least 7.0, such as at least 8.0, such as at least 9.0, such as at least 10.0, such as at least 15, such as at least 20, such as at least 30, such as at least 40, such as at least 50, such as at least 60, such as at least 70, such as at least 80, such as at least 90, such as at least 100, such as at least 100, such as at least 1000, such as at least 10000.

For all of the methods disclosed above R is the ratio of the concentration of lectin molecules having a high molecular weight above the molecular weight for dimer lectins to the concentration of lectin molecules having a low molecular weight below or equal to the molecular weight for dimer lectins, and in a preferred embodiment R is the ratio of the concentration of lectin molecules having a high molecular weight above the molecular weight for trimer lectins to the concentration of lectin molecules having a low molecular weight below or equal to the molecular weight for trimer lectins.

The lectin according to the invention, may be any lectin wherein several oligomeric forms are produced, such as mannose-binding lectins. In particular the invention relates to a method for producing MBL composition. By the term MBL is meant mannose-binding lectin (or mannose-binding protein, as denoted by some authors). MBL is preferably human MBL having a protein sequence as shown is for example PCT application WO00/70043 or derivatives or variants thereof being functional equivalents of MBL. In the following the invention will be described in relation to the lectin MBL.

The methods according to the present invention allow large scale manufacturing of the lectins in question. Thus, in another aspect the invention relates to any industrial or large scale method of manufacturing MBL comprising the application of said methods during the manufacturing of said MBL.

### DRAWINGS

FIG. 1: SDS-PAGE Western using HYB 131-01 raised against plasma derived MBL. P=Plasma derived MBL; 1=Recombinantly derived MBL before use of invention; 3=Recombinantly derived MBL after use of invention.

FIG. 2: SDS-PAGE Western using HYB 131-01 raised against plasma derived MBL. Left: Immunoblot, Right: Densiometric Analysis of Blot (Scion Software). Samples:

1=Recombinantly derived MBL before use of invention (R=6); molecular weight of dominant bands in this example are (from right to left) 47 kDa, 160 kDa, 230 kDa, 290 kDa, 330 kDa, and 365 kDa (shoulder).

2=Recombinantly derived MBL after use of invention (supernatant) (R=0); molecular weight of dominant bands in this example are (from right to left) 47 kDa and 160 kDa.

3=Recombinantly derived MBL after use of invention (precipitate) (R>>1000); molecular weight of dominant bands in this example are (from right to left) 230 kDa, 290 kDa, 330 kDa, and 365 kDa (shoulder) and 400-600 kDa.

FIG. 3: Precipitation of MBL in 3 L HyQ medium. Left: Medium before precipitation. Middle: Medium just after addition of precipitating agent. Right: Medium 40 min after addition of precipitating agent.

FIG. 4: Calculation examples of R-values using BioAnalyzer system. Left: Fluorescence stained gel (Agilent standard method, L=marker ladder). Right: Peak integrations of sample 1 and 3 (arrows indicate integrated peaks for calculation of R).

Samples:

1 and 2=Recombinantly derived MBL dominated by high molecular weight forms (R=19).

3 and 4=Recombinantly derived MBL dominated by low molecular weight forms (R=0.2)

### DETAILED DESCRIPTION OF THE INVENTION

The ratio R discussed herein may be calculated using any suitable method. In a preferred embodiment a quantitive estimate of the R value of a in a sample might be done as follows:

The SDS-PAGE immunoblot is scanned into a TIFF file, or another non-compressed bitmap file. The pixel density along the sample bands are measured in a picture evaluation program, such as Scion Image for Windows 4.0 (Scion Corporation, freeware beta version on www.scioncorp.com), and exported to a data evaluation program (like Excel).

The migration corresponding to the dimeric lectin is settled from the migration of markers (Precision Protein Standards, BioRad). The R value is calculated as the total pixel signal above reference migration point (for MBL 200 kDa), divided by the total pixel signal beneath the reference migration point (for MBL 200 kDa). R₀ is defined as the ratio in the starting material, while R₁ is the ratio along the corresponding evaluation line in the precipitated sample.

Another method for measuring the ratio R is to use a chip based electrophoresis system as a BioAnalyzer system (from Agilent). Here, integrations of peak areas are done as part of the standard evaluation procedure.

Precipitating Agent

It is a central aspect of the present invention that it is possible to separate low mass forms and high mass forms of MBL by precipitating an MBL preparation comprising both forms. The term precipitating agent is used synonymously with the term precipitant in this context. The precipitating agent may be any substantially non-denaturing, water-soluble protein precipitating agent, many of these well-known in the field of protein purification. Thus, the precipitating agent may be a high molecular mass precipitant, such as PEG with masses such as 200 Da, 300 Da, 400 Da, 550 Da, 600 Da, 1,000 Da, 1,450 Da, 1,500 Da, 2,000 Da, 3,000 Da, 3,350 Da, 4,000 Da, 6,000 Da, 8,000 Da, 10,000 Da, 15,000 Da, 20,000 Da, and 35,000 Da, and chemical conjugates of PEG with another compound, such as PEG itself or proteins, many of which are commercially available PEG compositions.

It is however preferred that the precipitating agent is a low mass agent. Examples of low mass agents are ammonium sulphate, sodium sulphate, potassium sulphate, sodium phosphates, and sodium chloride. Examples of effective anionic agents are ammonium (NH₄⁺), sodium ion (Na⁺), potassium ion (K⁺), and caesium (Cs⁺). Examples of effective cationic agents are sulphate (SO₄²⁻), phosphate (PO₄³⁻), and acetate (CH₂COO⁻).

Thus in one embodiment the precipitating agent is selected from cationic precipitating agents. The precipitating agent may be selected from Ca²⁺-containing agents, such as calcium chloride (CaCl₂, CaCl₂.H₂O, CaCl₂.2H₂O, CaCl₂.6H₂O), calcium nitrate (Ca(NO₃)₂, Ca(NO₃)₂.3H₂O, Ca(NO₃)₂.4H₂O), calcium nitrite (Ca(NO₂)₂.H₂O, Ca(NO₂)₂.4H₂O), calcium iodide (CaI₂, CaCl₂.6H₂O), calcium bromide (CaBr₂, CaBr₂.6H₂O), bromate (Ca(BrO₃)₂.H₂O), calcium chlorate (Ca(ClO₃)₂, Ca(ClO₃)₂.2H₂O, (CaClO₄)₂), calcium chromate (CaCrO₄.2H₂O), calcium permanganate (Ca(MnO₄)₂.5H₂O), calcium hypophosphite (Ca(H₂PO₂)₂), calcium iron cyanides (Ca₃[Fe(CN)₆]₂.12H₂O, Ca₂Fe(CN)₆.12H₂O), calcium thiosulphate (CaS₂O₃.6H₂O), and such as less soluble calcium containing agents, such as calcium formate (Ca(CHO₂)₂), calcium acetate (Ca(C₂H₃O₂)₂, Ca(C₂H₃O₂)₂.H₂O, Ca(C₂H₃O₂)₂.2H₂O) calcium propionate (Ca(C₃H₅O₂)₂.H₂O), calcium lactate (Ca(C₃H₅O₃)₂.5H₂O), calcium maleate (CaC₄H₂O₄.H₂O), calcium valerate (Ca(C₅H₉O₂)₂), and calcium citrate (Ca₃(C₆H₅O₇)₂.4H₂O).

A cationic precipitating agent is preferably added in a concentration in the range of from 0.001 M to 5.0 M, such as from 0.01 M to 5.0 M, such as from 0.05 M to 2.5 M, such as from 0.01 M to 2.5 M, for example 0.1 M to 2.0 M, such as from 0.1 M to 1.0 M.

In another embodiment the precipitating agent is selected from anionic precipitating agents. The anionic precipitating agent may be selected from anions, such as phosphates, carbonates, and sulphates.

The counter-ion for the precipitating agent may be present in the solvent of the preparation or may be added either prior to, simultaneously with, or after addition of the precipitating agent. In respect of cationic precipitating agents, the preparation preferably contains an anion selected from carbonates, sulphates and/or phosphates, preferably phosphates.

The separation of precipitate from supernatant may be conducted by any suitable method, preferably filtration or centrifugation.

The preparation may comprise a solvent, such as a solvent selected from a broth from a cell cultivation tank, a liquid intermediary product from a purification process, a liquid formulation, any buffered solution, or pure water. In a preferred embodiment the solvent is a culture medium, whereby the method may be applied directly to the culture broth after culturing cells expressing MBL optionally preceded by a filtration step.

Once the precipitate has formed it may be separated from the supernatant by any suitable procedure, in particular a procedure suitable for large scale production. The separation may be conducted by centrifugation followed by removal of the supernatant for example by suction. Thereby another advantage of the invention reveals, namely that the large amount of water from fermentation is removed without losing substantial amounts of the relevant MBL. Furthermore, as described above the supernatant comprising low mass molecules may be further processed for example by capturing the MBL molecules through chromatography.

Precipitation may be done at any pH, in which the formed precipitate is stable. Limits for pH is normally 2<pH<11, such as pH=3, for example pH=4, such as pH=5, for example pH=6, such as pH=7, for example pH=8, such as pH=9, for example pH=10.

Precipitation may be done at any temperature, in which the formed precipitate is stable. Limits for temperature setting are 0° C.<temperature<80° C., such as 4° C., for example 10° C., such as 15° C., for example 20° C., such as 25° C., for example 30° C., such as 35° C., for example 40° C., such as 50° C., for example 60° C., such as 70° C., however, the precipitation is normally carried out at room temperature.

The precipitating agent is preferably selected to that the precipitation may be done within a period of days, preferably within a period of hours, preferably within a period of 30 minutes.

Whenever referred to in this text the molecular weight is measured by SDS-PAGE, using a Novex system and commercially available gels (NuPAGE™ 3-8% Tris-Acetate, Invitrogen). MBL is recognized by immunoblotting using a commercially available MBL specific antibody (Hyb131-01, Statens Serum Institute, Copenhagen). Estimation of molecular mass of major Hyb131-01 bands is made by interpolation on standard curve based on BioRad Precision Protein Standards™ (BioRad 161-0362), by plotting ln(migration) versus mass.

Composition

The degree of increase of the ratio R depends on the starting material having ration R₀, the starting material being the MBL preparation. It is preferred that at least 50 mole % of the MBL in the composition has a molecular weight above the molecular weight for dimer lectins, preferably above the molecular weight for trimer lectins. It is thus preferred that at least 50 mole % of MBL in the composition has a molecular weight above 200 kDa, such as above 225 kDa, such as above 250 kDa, such as above 300 kDa.

By the term ‘mole %’ is meant the relative amount of moles of MBL polypeptide chains in the composition. However, mole % can also be expressed as the relative amount of moles of MBL subunits each comprising three MBL polypeptides. Alternatively, the percentage values disclosed herein can be calculated as weight percent values (% (w/w)). For the sake of clarity the percentage values are expressed herein as “mole %” in relation to MBL polypeptide chains, but the identical percentage values could equally well be expressed as mole % in relation to MBL subunits, or as weight percent values.

It appears as if the MBL obtained by the present method provides fractions comprising only the oligomers, without comprising denatured and/or structurally impaired MBL molecules normally seen in plasma MBL, since the bands on the SDS-PAGE substantially correspond to molecules failing in one or more of the following classes:
class I having molecular weight in the range of 200 kDa to 270 kDa (preferably comprising dimers and/or trimers),
class II having molecular weight in the range of from 270 kDa to 300 kDa (preferably comprising trimers and/or tetramers),
class III having molecular weight in the range of from 300 kDa to 400 kDa (preferably comprising tetramers and/or pentamers), and
class IV having molecular weight in the range of from 400 kDa to 600 kDa (preferably comprising pentamers and/or higher oligomers), said molecular weight preferably being determined by SDS-PAGE,
wherein MBL in class I constitutes an amount in the range of from 0-20 mole %, such as from 0-10 mole %, for example from 0-5 mole %, such as from 0-2 mole %, for example from 0-1 mole %, such as from 0 to less than 0.5 mole %, for example from 0 to less than 0.1 mole %, such as from 0 to less than 0.01 mole %, for example from 0 to less than 0.001 mole %, such as from 0.1-20 mole %, such as 0.1-10 mole %, for example from 0.1-5 mole %, such as from 0.1-2 mole %, for example from 0.1-1 mole %, such as from 0.1 to less than 0.5 mole %, for example from 0.1 to less than 0.4 mole %, such as from 0.1 to less than 0.3 mole %, for example from 0.1 to less than 0.2 mole % of the total amount of MBL polypeptide chains in the composition.

Preferably the MBL composition comprises molecules falling in at least two of the classes, such as molecules failing in at least three of the classes, more preferably in four of the classes.

When it is stated herein above that a certain class preferably comprises certain MBL oligomers this does not mean that oligomers other than the ones indicated for a particular class cannot also be present. However, without being bound by theory and according to one presently preferred hypothesis, it is believed that the oligomers listed above are mainly found in the classes for which they are stated.

By the term “substantially correspond” is meant that minor amounts of MBL having a molecular weight higher that 600 kDa may be present, such as minor amounts of below 10 mole %, such as below 5 mole %. Further, “substantially” also refers to any deviation within 5%, such as 2%, for example 1%, such as 0.5%, for example less than 0.1% in the molecular weights stated for each class.

More preferably MBL in class I constitutes an amount in the range of from 0.1-15 mole % of the total amount of MBL in the composition, such as an amount in the range of from 0.1-12 mole % of the total amount of MBL in the composition, such as an amount in the range of from 0.1-10 mole % of the total amount of MBL in the composition, such as an amount in the range of from 0.1-5 mole % of the total amount of MBL in the composition.

Preferably MBL in class II constitutes an amount in the range of from 0-50 mole % of the total amount of MBL in the composition, such as an amount in the range of from 0-30 mole % of the total amount of MBL in the composition, such as an amount in the range of from 5-30 mole % of the total amount of MBL in the composition, such as an amount in the range of from 10-30 mole % of the total amount of MBL in the composition.

Preferably MBL in class III constitutes an amount in the range of from 0-60 mole % of the total amount of MBL in the composition, such as an amount in the range of from 10-60 mole % of the total amount of MBL in the composition, such as an amount in the range of from 20-60 mole % of the total amount of MBL in the composition, such as an amount in the range of from 20-50 mole % of the total amount of MBL in the composition.

Preferably MBL in class IV constitutes an amount in the range of from 0-50 mole % of the total amount of MBL in the composition, such as an amount in the range of from 0-30 mole % of the total amount of MBL in the composition such as an amount in the range of from 5-30 mole % of the total amount of MBL in the composition, such as an amount in the range of from 10-30 mole % of the total amount of MBL in the composition.

In preferred embodiments the composition obtained by the present invention comprises MBL of class I in an amount of from 0-5 mole %, MBL of class II in an amount of from 10-30 mole %, MBL of class III in an amount of from 20-50 mole %, and MBL of class IV in an amount of from 10-30 mole %, such as MBL of class I in an amount of from 0-5 mole %, MBL of class II in an amount of from 15-30 mole %, MBL of class III in an amount of from 20-50 mole %, and MBL of class IV in an amount of from 15-30 mole %, for example MBL of class I in an amount of from 0-5 mole %, MBL of class II in an amount of from 20-30 mole %, MBL of class III in an amount of from 20-50 mole %, and MBL of class IV in an amount of from 20-30 mole %, such as MBL of class I in an amount of from 0-5 mole %, MBL of class II in an amount of from 20-30 mole %, MBL of class III in an amount of from 30-50 mole %, and MBL of class IV in an amount of from 20-30 mole %.

For the above compositions, the MBL of class I can be absent or present in an amount of e.g. 0.1 mole %, such as 0.2 mole %, for example 0.4 mole %, such as about 1 mole %, for example about 2 mole %, such as about 3 mole %, for example about 4 mole %. “About” shall be understood to indicate any deviation below 5%, such as 2%, for example 1%, such as 0.5%, for example less than 0.1% in the amount of MBL present in class I.

In a preferred embodiment the classes are as defined below:
class I having molecular weight in the range of 200 kDa to 260 kDa,
class II having molecular weight in the range of from 270 kDa to 300 kDa,
class III having molecular weight in the range of from 310 kDa to 390 kDa, and
class IV having molecular weight in the range of from 400 kDa to 600 kDa, said molecular weight being determined by SDS-PAGE,

The determination of the percent of MBL in each class I to IV is conducted as described above for the determination of the ratio R.

In another aspect of the invention there is provided a method for obtaining a composition comprising MBL selected from high molecular weight MBL and low molecular weight MBL, wherein said composition comprises less than 20 mole % of low molecular weight MBL, said low molecular weight MBL being of a molecular weight less than 300 kDa.

Accordingly, the present invention also relates to a composition comprising high molecular weight MBL and low molecular weight MBL, wherein said composition comprises less than 20 mole % of low molecular weight MBL, said low molecular weight MBL being of a molecular weight less than 300 kDa.

Accordingly, there is provided methods and compositions as stated herein immediately above, wherein the low molecular weight MBL amounts to less than 10 mole %, such as less than 5 mole %, for example less than 2 mole %, such as less than 1 mole %, for example less than 0.1 mole %, such as less than 0.01 mole %, for example less than 0.001 mole %; as well as any of the aforementioned compositions wherein said low molecular weight MBL preferably has a molecular weight of less than 300 kDa, for example less than 290 kDa, such as less than 285 kDa, for example less than 280 kDa, such as less than 275 kDa, for example less than 270 kDa, such as less than 265 kDa, for example less than 260 kDa, such as less than 255 kDa, for example less than 250 kDa, such as less than 245 kDa, for example less than 240 kDa, such as less than 235 kDa, for example less than 230 kDa, such as less than 225 kDa, for example less than 220 kDa, such as less than 215 kDa, for example less than 210 kDa, such as less than 205 kDa, for example less than 200 kDa.

By producing an MBL composition according to the present invention, the MBL composition is substantially free from any impurities naturally associated with the MBL when produced in a native host organism, such as plasma MBL.

The method may be used for producing high mass MBL compositions from any MBL source, such as any mammalian recombinant MBL. It is however preferred that the MBL of the composition is human, such as MBL wherein the MBL subunit is assembled of three peptide sequences comprising the sequence as shown i SEQ ID NO:1 in PCT application WO00/70043 or a functional equivalent thereof.

In a further aspect of the present invention the two purified fractions of high and low molecular weight MBL, respectively, can be pooled in any desirable ratios of each fraction.

Recombinant Production

Although the method may be applied to any MBL starting material having both low and high mass MBL, it is particularly suitable for producing high mass MBL from a recombinantly produced preparation.

Thus, the MBL preparation is preferably a recombinant preparation, wherein the MBL preparation is obtained by
preparing a gene expression construct encoding human MBL peptide or a functional equivalent thereof,
transforming a host cell culture with the construct,
cultivating the host cell culture in a culture medium, thereby obtaining expression and secretion of the polypeptide into the culture medium,
obtaining a preparation comprising a variety of MBL molecules

According to the invention, the sequences from the MBL gene may be from the human MBL gene or from MBL genes of other animal species, in which the immune system in this respect is acting like the human immune system. An example of a preferred embodiment of a preparation of a recombinant MBL according to the invention is described in example 1 of PCT application WO00/70043 which is incorporated herein by reference. In the example the recombinant MBL is prepared by the use of an expression vector comprising sequences from the human MBL gene.

The invention also concerns the use of expression vectors comprising sequences, which are functional derivatives of the sequences of the human MBL gene. By said functional derivatives are meant sequences, which contain base pair alterations that lead to no functional or essentially no functional differences of the expression vector and the in this way prepared MBL has a functionality comparable to the MBL prepared by the use of an expression vector comprising the unaltered sequences from the human MBL gene.

In addition to the purification method it is preferred that the gene expression construct and the host cell also favours production of higher oligomers. Accordingly, the gene expression construct preferably comprises at least one intron sequence from the human MBL gene or a functional equivalent thereof. Furthermore, the gene expression construct may comprise at least two exon sequences from the human MBL gene or a functional equivalent thereof. More preferably the gene expression construct comprises at least three exon sequences from the human MBL gene or a functional equivalent thereof. When comprising more than one exon, the exon sequences are preferably aligned as in the human MBL gene.

Although preferred that the sequence comprises intron sequences, it may for some applications be convenient that the expression construct comprises a cDNA sequence encoding a MBL subunit or a functional equivalent thereof.

The invention features the use of MBL gene expression constructs rather than MBL cDNA constructs for expression of rMBL in mammalian cell lines or transgenic animals to obtain recombinant MBL with structural properties under non-denaturing and denaturing conditions being substantially similar to natural human MBL. By “recombinant human MBL” is meant human MBL which is expressed from engineered nucleic acids and by “MBL gene expression constructs” is meant an expression vector suitable for expression in mammalian cell lines, which contains exon sequences and at least one intron sequence from the human MEL gene or from MBL genes of other animal species, such as but not limited to chimpanzees and rhesus monkeys.

Preferably, the DNA sequences encode a polypeptide sequence as shown in SEQ ID NO: 1 or a functional equivalent, whereby a functional equivalent is as defined above. SEQ ID NO:1 corresponds to the MBL sequence having database accession NO: P11226. The equivalent may be obtained by a modification of the peptide sequence shown as SEQ ID NO: 1, such as a sequence processing a corresponding property as the sequences mentioned in the present invention, but wherein one or more amino acids have been substituted with others. Preferably a functional equivalent contains conservative substitutions, i.e. where one or more amino acids are substituted by an amino acid having similar properties, such that a person skilled in the art of protein chemistry will expect the secondary and tertiary structure of the protein to be unchanged. Amino acids suitable for conservative substitutions include those having functionally similar side chains. For example, hydrophobic residues: e.g. glycine, alanine, valine, leucine, isoleucine and methionine may replace another such residue. Similarly, conservative substitutions may involve interchanging hydrophilic residues: (e.g.: arginine and lysine, glutamine and aspargine, threonine and serine), basic reduces (e.g., lysine, arginine and histidine), and/or acidic residues (e.g., aspartic acid and glutamic acid). Functional equivalents may also, or alternatively, be modified by for example the deletion or addition of amino acids, or the chemical modification of amino acids, as long as the function of the polypeptide is preserved.

The isolated MBL peptide including any functional equivalents thereof, may in one embodiment comprise at least 80 amino acid residues, such as at least 100 amino acid residues, such as at least 150 amino acid residues, such as at least 200 amino acid residues, for example at least 220 amino acid residues, such as at least 250 amino acid residues.

In a preferred embodiment the expression vector is suitable for expression in mammalian cell lines or transgenic animals, which contains exon sequences and at least one intron sequence from the human MBL gene or from MBL genes of other animal species, such as, but not limited to, chimpanzes and rhesus monkeys. In one embodiment the host cell culture is cultured in a transgene animal. By a transgenic animal in this context is meant an animal which has been genetically modified to contain and express the human MBL gene or fragments or mimics hereof.

In a preferred embodiment the expression construct of the present invention comprises a viral based vector, such as a DNA viral based vector, a RNA viral based vector, or a chimeric viral based vector. Examples of DNA viruses are cytomegalo virus, Herpex Simple, Epstein-Barr virus, Simian virus 40, Bovine papillomavirus, Adeno-associated virus, Adenovirus, Vaccinia virus, and Baculo virus.

In mammalian host cells, a number of viral-based expression systems may be utilized. In cases where an adenovirus is used as an expression vector, the nucleic acid molecule of the invention may be ligated to an adenovirus transcription/translation control complex, for example, the late promoter and tripartite leader sequence. This chimeric gene may then be inserted in the adenovirus genome by in vitro or in vivo recombination. Insertion in a non-essential region of the viral genome (for example, region E1 or E3) will result in a recombinant virus that is viable and capable of expressing a MASP-3 gene product in infected hosts (for example, see Logan and Shenk, Proc. Natl. Acad. Sci. USA 81:3655-3659, 1984). Specific initiation signals may also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544, 1987).

Examples of, RNA virus are Semliki Forest virus, Sindbis virus, Poko virus, Rabies virus, Influenza virus, SV5, Respiratory Syncytial virus, Venezuela equine encephalitis virus, Kunjin virus, Sendai virus, Vesicular stomatitisvirus, and Retroviruses.

Examples of chimetic viruses are Adeno-virus, Sindbis virus and Adenovirus adeno-associated virus.

Regarding specific vectors reference is made to Makrides, S. C., “Components of vectors for Gene Transfer and Expression in Mammalian Cells”, which is hereby incorporated by reference.

In particular, an Epstein-Barr virus origin of replication or functional derivatives or mimics hereof including the pREP9 vector is used.

In one aspect the invention provides an expression construct encoding human MBL, featured by comprising one or more intron sequences from the human MBL gene including functional derivatives hereof. Additionally, it contains a promoter region selected from genes of virus or eukaryotes, including mammalia and insects.

The promoter region is preferably selected to be different from the human MBL promoter, and preferably in order to optimize the yield of MBL and size distribution of MBL oligomers, the promoter region is selected to function most optimally with the vector and host cells in question.

In a preferred embodiment the promoter region is selected from a group comprising Rous sarcoma virus long terminal repeat promoter, and cytomegalovirus immediate-early promoter, and elongation factor-1 alpha promoter.

In another embodiment the promoter region is selected from genes of microorganisms, such as other viruses, yeasts and bacteriae.

In order to obtain a greater yield of recombinant MBL, the promoter, region may comprise enhancer elements, such as the QBI SP163 element of the 5′ end untranslated region of the mouse vascular endothelia growth factor gene. The construct is used for transforming a host cell to obtain a host cell culture capable of expressing MBL. The host cell culture is preferably an eucaryotic host cell culture. By transformation of an eukaryotic cell culture is in this context meant introduction of recombinant DNA into the cells. The expression construct used in the process is characterised by having the MBL encoding region selected from mammalian genes including human genes and genes with big resemblance herewith such as the genes from the chimpanzee. The expression construct used is furthermore featured by the promoter region being selected from genes of virus or eukaryotes, including mammalian cells and cells from insects.

The process for producing recombinant MBL according to the invention is characterised in that the host cell culture is preferably eukaryotic, and for example a mammalian cell culture. A preferred host cell culture is a culture of human kidney cells and in an even more preferred form the host cell culture is a culture of human embryonal kidney cells (HEK cells). The invention features the use of HEK 293 cell lines for production of recombinant human MBL. By “HEK 293 cell lines” is meant any cell line derived from human embryonal kidney tissue such as, but not limited to, the cell lines deposited at the American Type Culture Collection with the numbers CRL-1573 and CRL-10852.

Other cells may be chick embryo fibroblasts, hamster ovary cells, baby hamster kidney cells, human cervical carcinoma cells, human melanoma cells, human kidney cells, human umbilical vascular endothelium cells, human brain endothelium cells, human oral cavity tumor cells, monkey kidney cells, mouse fibroblasts, mouse kidney cells, mouse connective tissue cells, mouse oligodendritic cells, mouse macrophage, mouse fibroblast, mouse neuroblastoma cells, mouse pre-B cells, mouse B lymphoma cells, mouse plasmacytoma cells, mouse teratocacinoma cells, rat astrocytoma cells, rat mammary epithelium cells, COS, CHO, BHK, 293, VERO, HeLa, MDCK, WI38, and NIH 3T3 cells.

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. The mammalian cell types listed above are among those that could serve as suitable host cells.

The host cell culture may be cultured in any suitable culture medium. Example of culture medium are RPMI-1640 or DMEM supplemented with, e.g., insulin, transferrin, selenium, and foetal bovine serum.

Purification

As discussed above the present invention allows a fast production of MBL. The production of MBL may be conducted by the following steps:

Fermentation—culturing of MBL expressing cells

Separation—precipitation followed by separation of precipitate and supernatant

Optionally purification—

Accordingly, the MBL composition may be purified by any suitable means, such as by any physico-chemical isolating method, including but not limited to filtration methods, chromatography, such as ion-exchange based on charge, gel permeation based on size, hydrophobic interaction based on hycrophobicity, or affinity chromatography.

The recombinant MBL composition is preferably purified from the fractions by means of affinity chromatography, such as mannose affinity chromatography.

Functionality

The functionality of the recombinant MBL composition obtained according to the invention is preferably resembling the functionality of plasma or serum MBL. In the present context the functionality of MBL is meaning the capability of activating the complement system as discussed above in relation to functional equivalents. The functionality may be expressed as the specific activity of MBL, such as units of MBL activity per ng MBL. The functionality the recombinant MBL composition as expressed as specific activity is preferably at least 25% of the specific activity of MBL purified from serum, such as at least 50% of the specific activity of MBL purified from serum, more preferred at least 75% of the specific activity of MBL purified from serum.

The functionality of MBL may be estimated by its capacity to form an MBL/MASP complex leading to activation of the complement system. When C4 is cleaved by MBL/MASP an active thiol ester is exposed and C4 becomes covalently attached to nearby nucleophilic groups. A substantial part of the C4b will thus become attached to the coated plastic well and may be detected by anti-C4 antibody.

A quantitative TRIFMA for MBL functional activity was constructed by 1) coating microtitre wells with 1 mg mannan in 100 ml buffer; 2) blocking with Tween-20; 3) applying test samples, e.g. diluted MBL preparations 4) applying MBL deficient serum (this leads to the formation of the MBL/MASP complex); alternatively the MBL and the MBL deficient serum may be mixed before application with the microtitre wells; 5) applying purified complement factor C4 at 5 mg/ml; 6) incubate for one hour at 37° C.; 7) applying Eu-labelled anti-C4 antibody; 8) applying enhancement solution; and 9) reading the Eu by time resolved fluorometry. Between each step the plate is incubated at room temperature and washed, except between step 8 and 9.

Estimation by ELISA may be carried out similarly, e.g. by applying biotin-labeled anti-C4 in step 7; 8) apply alkaline phosphatase-labeled avidin; 9) apply substrate; and 10) read the colour intensity. A calibration curve can be constructed using dilutions of one selected normal plasma. In relation to the present invention the following serum is an example of useful serums: plasma pool LJ 6.57 Apr. 28, 1997. The functionality may be expressed as the specific activity of MBL, such as in units of MBL activity per ng MBL.

Another assay for determining a functional equivalent to MBL is to determine the ability to bind to receptor/receptors on cells.

The interaction of MBL with receptor/receptors on cells may be analysed by cytofluorimetry. 1) MBL at a concentration of 50 μg/ml is incubated with 2×105 cells. The binding is carried out in phosphate buffered salt solution (PBS) containing 1% FCS and 0.1% Na-azide. 2) For detection of cell-bound MBL, biotinylated anti-MBL antibody is applied; 3) followed by the addition of strepavidin-FITC and 4) analysis of the mixture by fluorimetry.

Pharmaceutical Composition

The composition obtained may be used as such for preparing a pharmaceutical composition, or the composition may be subjected to further purification steps before being used as discussed above.

The MBL composition obtained by the present invention may be used for the preparation of a pharmaceutical composition for the prevention and/or treatment of various diseases or conditions.

In addition to the MBL oligomers, the pharmaceutical composition may comprise a pharmaceutically acceptable carrier substance and/or vehicles.

In particular, a stabilising agent may be added to stabilise the MBL proteins. The stabilising agent may be a sugar alcohol, saccharide, protein and/or amino-acids. An example of a stabilising agent may be albumin or maltose.

Other conventional additives may be added to the pharmaceutical composition depending on administration form for example.

In one embodiment the pharmaceutical composition is in a form suitable for injections. Conventional carrier substances, such as isotonic saline, may be used.

In another embodiment the pharmaceutical composition is in a form suitable for pulmonal administration, such as in the form of a powder for inhalation or creme or fluid for topical application.

The treatment needs not be a treatment of a diagnosed disease, disorder or condition in a presently or apparently need of treatment but may be used to prevent the disease or condition to occur.

A treatment in this context may comprise cure and/or prophylaxis of e.g. the immune system and reproductive system by humans and by animals having said functional units acting in this respect like those in humans. By conditions to be treated are not necessarily meant conditions presently known to be in a need of treatment, but comprise generally any condition in connection with current and/or expected need or in connection with an improvement of a normal condition. In particular, the treatment is a treatment of a condition of deficiency of MBL.

In another aspect of the present invention the manufacture is provided of a medicament consisting of said pharmaceutical compositions of MBL, including compositions of rMBL fragments or mimics hereof intended for treatment of conditions comprising cure and/or prophylaxis of conditions of diseases and disorders of e.g. the immune system and reproductive system by humans and by animals having said functional units acting like those in humans.

Said diseases, disorders and/or conditions in need of treatment with the compounds of the invention comprise eg treatment of conditions of deficiency of MBL, treatment of cancer and of infections in connection with immunosuppressive chemotherapy including in particular those infections which are seen in connection with conditions during cancer treatment or in connection with implantation and/or transplantation of organs. The invention also comprises treatment of conditions in connection with recurrent miscarriage.

Thus, in particular the pharmaceutical composition may be used for the treatment and/or prevention of clinical conditions selected from infections, MBL deficiency, cancer, disorders associated with chemotherapy, such as infections, diseases associated with human immunodeficiency virus (HIV), diseases related with congenital or acquired immunodeficiency. More particularly, chronic inflammatory demyelinating polyneuropathy (CIDP), Multifocal motoric neuropathy, Multiple scelrosis, Myasthenia Gravis, Eaton-Lambert's syndrome, Opticus Neuritis, Epilepsy; Primary anti-phosholipid syndrome; Rheumatoid arthritis, Systemic Lupus erythematosus, Systemic scleroderma, Vasculitis, Wegner's granulomatosis, Sjøgren's syndrome, Juvenile rheumatiod arthritis; Autoimmune neutropenia, Autoimmune haemolytic anaemia, Neutropenia; Crohn's disease, Colitis ulcerous, Coeliac disease; Asthma, Septic shock syndrome, Chronic fatigue syndrome, Psoriasis, Toxic shock syndrome, Diabetes, Sinuitis, Dilated cardiomyopathy, Endocarditis, Atherosclerosis Primary hypo/agammaglobulinaemia including common variable immunodeficiency, Wiskot-Aldrich syndrome and serve combined immunodefiency (SCID), Secondary hypo/agammaglobulinaemia in patients with chronic lymphatic leukaemia (CLL) and multiple myeloma, Acute and chronic idiopathic thrombocytopenic purpura (ITP), Allogenic bone marrow transplantation (BTM), Kawasaki's disease, and Guillan-Barre's syndrome.

The route of administration may be any suitable route, such as intravenously, intramusculary, subcutaneously or intradermally. Also, pulmonal or topical administration is envisaged by the present invention.

In particular the MBL composition may be administered to prevent and/or treat infections in patients having clinical symptoms associated with congenital or acquired MBL deficiency or being at risk of developing such symptoms. A wide variety of conditions may lead to increased susceptibility to infections in MBL-deficient individuals, such as chemotherapy or other therapeutic cell toxic treatments, cancer, AIDS, genetic disposition, chronic infections, and neutropenia.

It appears that cancer patients treated by chemotherapy are often susceptible to infection due to adverse effects of the drug regime on cells of the immune system, which is the background for the use of MBL therapy in the treatment of this condition. The observed low plasma concentrations of MBL (below 500 ng/mL) are indicative for an increased susceptibility to clinical significant infections, and the immune defense of these patients can be reinforced by administration of recombinant or natural plasma-derived MBL.

The pharmaceutical composition may thus be administered for a period before the onset of administration of chemotherapy or the like and during at least a part of the chemotherapy.

The MBL composition may be administered as a general “booster” before chemotherapy, or it may be administered to those only being at risk of developing MBL deficiency. The croup of patients being at risk may be determined be measuring the MBL level before treatment and only subjecting those to treatment whose MBL level is below a predetermined level. The limit for determining a low MBL level is evaluated to be below 500 ng/ml for most groups. The MBL level may be determined by time resolved immunofluorescent assay as described in Example 9, ELISA, RIA or nephelometry.

Another indication for administering MBL is when the MBL level is below 50% of the normal level, such as below 300 ng/ml, or below 200 ng/ml.

The MBL composition is administered in suitable dosage regimes, in particular it is usually administered at suitable intervals, eg. once or twice a week during chemotherapy.

Normally from 1-100 mg is administered per dosage, such as from 2-10 mg, mostly from 5-10 mg per dosage. Mostly about 0.1 mg/kg body weight is administered.

Thus, in one aspect the invention concerns MBL, including rMBL, fragments or mimics hereof for use in the treatment of cancer and of conditions of diseases and disorders of e.g. the immune system and reproductive system, said treatment consisting of creation, reconstitution, enhancing and/or stimulating the opsonic and/or bactericidal activity of the complement system, i.e. enhancing the ability of the immune defence to recognise and kill microbial pathogens.

Furthermore, an aspect of the present invention is the use of a recombinant composition according to the present invention in a kit-of-parts further comprising another medicament. In particular the other medicament may be an anti-microbial medicament, such as antibiotics.

Concerning miscarriage, it has been reported that the frequency of low plasma levels of MBL is increased in patients with otherwise not explained recurrent miscarriages, which is the background for lowering of the susceptibility to miscarriage by a reconstitution of the MBL level by administration of recombinant MBL in these cases.

As to the nature of compounds of the invention, it appears, that in its broad aspect, the present invention relates to compounds which are able to act as opsonins, that is, able to enhance uptake by macrophages either through direct interaction between the compound and the macrophage or through mediating complement deposition on the target surface. A particular example hereof is MBL, a fragment or a mimic hereof. The present invention is based upon the disclosure of a synthesis of a recombinant human MBL which appears to be closer to the structure of the natural human MBL than achieved in the past.

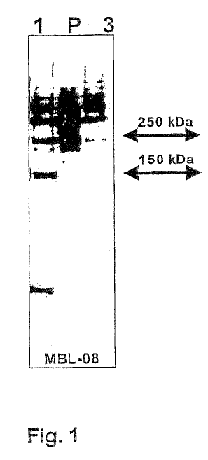

The invention has now been explained and accounted for in various aspects and in adequate details, but additionally it will be illustrated below by FIGS. 1 to 4 and the non-limiting examples of preferred embodiments.

### EXAMPLE 1

### Isolation of MBL from a Spinner Bottle Cultivation

MBL containing cell culture broth was acquired from continuous 200 mL spinner bottle cultivation: The MBL producing HEK293 cell line produced as described in Example 1 of WO 00/70043 was incubated in HyQ PF293 medium (HyClone) with G418 (CalBiochem), ascorbic acid (ICN Biomedicals) and CytoPore1 microcarriers (AP Biotech).

Withdrawal of 160-175 mL cell culture broths was done repeatedly, and after removal of cells by microfiltration, the cell free broth was applied for precipitation. After addition of CaCl₂ to the cell free broth with pH7-8 to obtain a Ca²⁺ concentration of 0.1 M, a white heavy precipitate is formed.

The precipitate might be resuspended into an EDTA containing solution at pH3.25. After dialysis, the dissolved MBL could be analysed by MBL assay (TRIFMA based on mannan binding and HYB131-01 recognition) and SDS-PAGE (Westerns using HYB131-01).

MBL specific immunoassays of the re-suspended precipitate reveal that a significant amount of the total MBL is recovered in the precipitate. Moreover, MBL recovered in the precipitate is dominated by oligomers larger than approximately 200 kDa (FIG. 1).

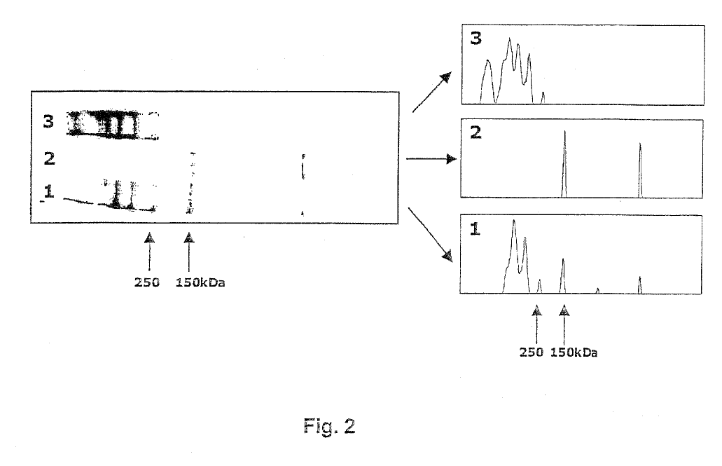

The same assay reveals that the corresponding supernatant reveals that some MBL is found in the supernatant. Moreover, MBL recovered in the supernatant is dominated by oligomers smaller than approximately 200 kDa, as seen in SDS-PAGE (FIG. 2).

### EXAMPLE 2

### Isolation of MBL from a Cultivation Tank

MBL containing cell culture broth was acquired from continuous 5 L tank cultivation: The MBL producing HEK293 cell line produced as described in Example 1 is incubated in HyQ PF293 medium (HyClone) with G418 (CalBiochem), glutamax (Gibco), ascorbic acid (ICN Biomedicals), and HySoy (HyClone).

Withdrawal of 200 mL cell culture broths was done repeatedly, and after removal of cells by centrifugation, the cell free broth was applied for precipitation.

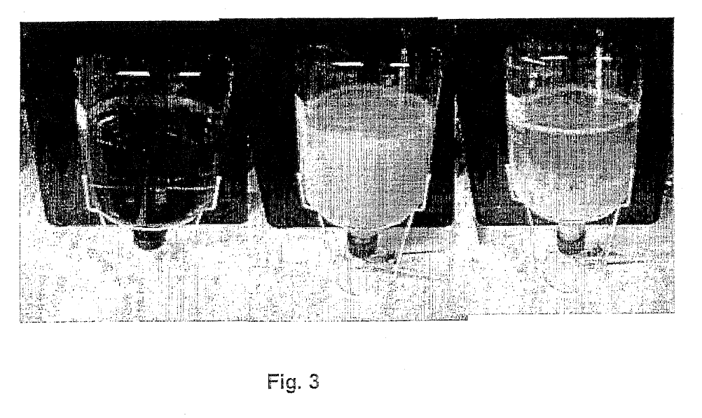

To assist the subsequent precipitation, sodium phosphate was added to obtain a phosphate concentration of approximately 200 mM. After addition of CaCl₂ to the phosphate enriched cell free broth to obtain a Ca²⁺ concentration of 0.1 M, a white heavy precipitate is formed. (See FIG. 3)

The precipitate might be re-suspended into a citrate containing solution at pH3.2.

### EXAMPLE 3

### Isolation of MBL from a SFMII Medium

MBL containing cell culture broth is acquired from continuous 5 L tank cultivation: The MBL producing HEK293 cell line is incubated in 293 SFMII medium (Gibco) with G418 (CalBiochem), glutamax (Gibco), and ascorbic acid (ICN Biomedicals). The cultivation is done as a draw-fill process with regular settling and change of medium. Microcarriers (Cytopore 1, AP Biotech) is used as support.

Withdrawal of 2 L cell culture broths is done repeatedly, and after removal of cells by centrifugation, the cell free broth is applied for precipitation.

To assist the subsequent precipitation, sodium phosphate is added to obtain a phosphate concentration of approximately 50 mM. After addition of CaCl₂ to the phosphate enriched cell free broth to obtain a Ca²⁺ concentration of 0.1 M, a white heavy precipitate is formed.

The precipitate is re-suspended into a citrate containing solution at pH 3.0.

### EXAMPLE 4

### Isolation of MBL from Non-Culture Medium

MBL containing solution (1.0 mg/L) is added sodium phosphate buffer (pH 7.4) to obtain a phosphate concentration of 100 mM. Subsequently, calcium chloride is added to obtain a calcium concentration of 100 mM. The formed precipitate contains most MBL (>90%), as shown by the MBL assay (TRIFMA based on mannan binding and HYB131-01 recognition) and SDS-PAGE (Westerns using HYB131-01).

### EXAMPLE 5

### Yields of MBL

Yields of mannan-binding MBL can be quantified in an MBL assay (TRIFMA based on mannan binding and HYB131-01 recognition). In two subsequent experiments, the MBL content in the resuspended precipitate of obtained in Example 1 in relation to the starting material was estimated to be 72% and 68%, respectively.

### EXAMPLE 6

### Calculation of the Ratio R

Three samples, corresponding to recombinantly derived MBL before use of invention, recombinantly derived MBL after use of invention (supernatant), and recombinantly derived MBL after use of invention (precipitate) were investicated to calculate R-values.

An SDS-PAGE immunoblot was prepared (FIG. 2), using MAB131-01 (Statens Serum Institute), rabbit anti mouse antibodies conjugated to horseradish peroxidase (P0260, Dako) and SuperSignal West Pico substrate (Pierce), and scanned into a non-compressed TIFF file. The pixel density along the sample bands was measured in Scion Image for Windows 4.0, and exported to Microsoft Excel.

The migration corresponding to 200 kDa was settled from the migration of markers (Precision Protein Standards, BioRad). The total pixel signal above 200 kDa was calculated, as was the total pixel signal beneath the 200 kDa marker. The ratio between these numbers in each sample represents the R-values.

The R-value in the recombinantly derived MBL before use of invention was estimated to be R=6. The R-value in the supernatant of recombinantly derived MBL after use of invention was estimated to be R=0, while the R-value in the precipitate of recombinantly derived MBL after use of invention was estimated to be R>>1000.

### EXAMPLE 7

### Calculation of the Ratio R

The R-value in a recombinantly derived MBL can be estimated using a BioAnalyzer System (Agilent) with Protein 200 Plus chip detection system (Agilent).

The samples are run exactly as described by the vendor—with the exception that the gel is diluted with water before the run (1 part water to 2 parts of gel)—and without adding reducing agent to the sample. This is to be able to see the high mass forms of MBL in the gel.

The migration corresponding to 200 kDa was settled from the migration of upper marker (myosin with molecular weight of 210 kDa, internal standard delivered by the vendor). The total pixel signal above 200 kDa was integrated using the standard integration routine, as was the total pixel signal beneath 200 kDa. The ratio between these numbers in each sample represents the R-values.

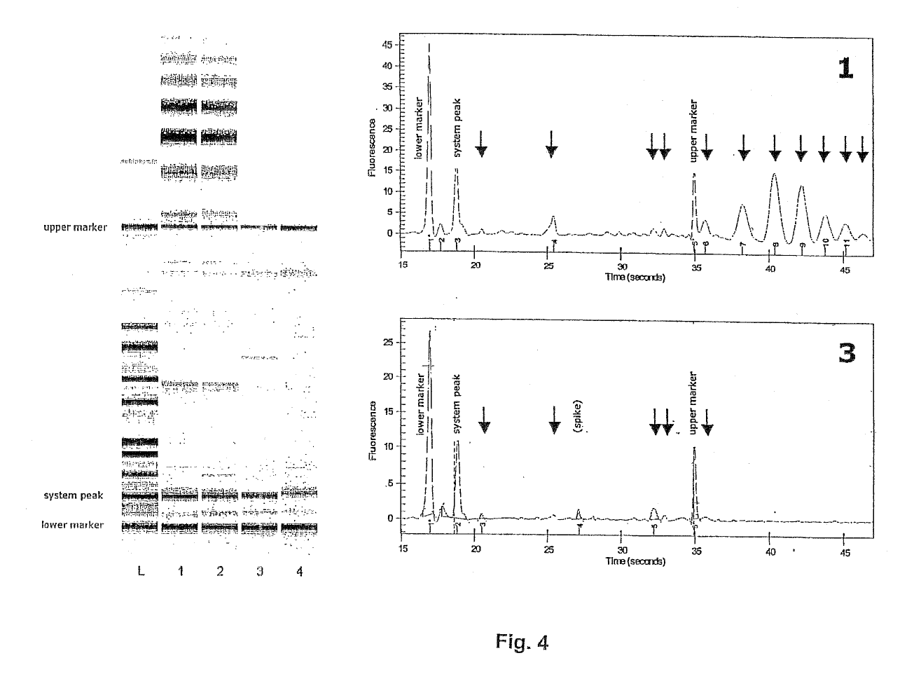

The calculation of the R-value in two selected samples of recombinantly derived MBL is shown in FIG. 4. The R-value in one sample is R=19, while the R-value in another sample is R=0.2

1. A method of separating high molecular weight mannose-binding lectin (MBL) molecules from low molecular weight MBL molecules of a MBL preparation comprising both high and low molecular weight recombinant MBL molecules, wherein high molecular weight MBL molecules are MBL molecules with a molecular weight above the molecular weight of MBL dimers, and low molecular weight MBL molecules are MBL molecules with a molecular weight equal or below the molecular weight of MBL dimers, said method comprising:
a) providing a MBL preparation comprising both high and low molecular weight recombinant MBL molecules, wherein more than 5 mole percent of the MBL molecules in said preparation are low molecular weight MBL molecules.
b) adding to said preparation a precipitating agent, wherein the precipitating agent is selected from cationic precipitating agents, and allowing a precipitate and a supernatant to form, wherein less than 5 mole percent of the MBL molecules of said precipitate are low molecular weight MBL molecules; and
c) separating said precipitate from said supernatant thereby obtaining a precipitate fraction and a supernatant fraction and thus separating high molecular weight MBL from low molecular weight MBL.

2. The method according to claim 1, wherein said precipitate is resuspended before obtaining said composition comprising high molecular weight MBL.

3. A method of separating high molecular weight mannose-binding lectin (MBL) molecules from low molecular weight MBL molecules of a MBL preparation comprising both high and low molecular weight recombinant human MBL molecules, wherein high molecular weight MBL molecules are MBL molecules with a molecular weight above the molecular weight of MBL dimers, and low molecular weight MBL molecules are MBL molecules with a molecular weight equal or below the molecular weight of MBL dimers, said method comprising
a) providing a MBL preparation comprising both high and low molecular weight recombinant MBL molecules, wherein more than 5 mole percent of the MBL molecules in said preparation are low molecular weight MBL molecules.
b) adding to said preparation a precipitating agent, wherein the precipitating agent is selected from cationic precipitating agents, and allowing a precipitate and a supernatant to form, wherein less than 5 mole percent of the MBL molecules of said precipitate are low molecular weight MBL molecules; and
c) separating said precipitate from said supernatant thereby obtaining a precipitate fraction and a supernatant fraction and thus separating high molecular weight MBL from low molecular weight MBL.
wherein MBL with a molecular weight below the molecular weight of MBL dimers comprises MBL having a molecular weight below 200 kDa.

4. The method according to claim 1, wherein the precipitating agent is selected from Ca2+ containing precipitating agents.

5. The method according to claim 4, wherein the precipitating agent is selected from the group consisting of CaCl₂, calcium chloride (CaCl₂, CaCl₂.H₂O, CaCl₂.2H₂O, CaCl₂.6H₂O) calcium nitrate (Ca(NO₃)₂, Ca(NO₃)₂.3H₂O, Ca(NO₃)₂.4H₂O), calcium nitrite (Ca(NO₂)₂.H₂O, Ca(NO₂)₂.4H₂O), calcium iodide (CaI₂, CaI₂.6H₂O), calcium bromide (CaBr₂, CaBr₂.6H₂O), bromate (Ca(BrO₃.H₂O), calcium chlorate (Ca(ClO₃)₂, Ca(ClO₃)₂.2H₂O, (CaClO₄)₂), calcium chromate (CaCrO₄.2H₂O), calcium permanganate (Ca(MnO₄)₂.5H₂O), calcium hypophosphite (Ca(H₂PO₂)₂), calcium iron cyanides (Ca₃[Fe(CN)₆]₂.12H₂O, Ca₂Fe(CN)₆.12H₂O), calcium thiosulphate (CaS₂O₃.6H₂O), calcium formate (Ca(C₃H₅O₃)₂.5H₂O), calcium acetate (Ca(C₂H₃O₂)₂, Ca(C₂H₃O₂)₂.H₂O, Ca(C₂H₃O₂)₂.2H₂O), calcium propionate (Ca(C₃H₅O₂)₂.H₂O), calcium lactate (Ca(C₃H₅O₃)₂.5H₂O), calcium maleate (CaC₄H₂O₄.H₂O), calcium valerate (Ca(C₅H₉O₂)₂), and calcium citrate (Ca₃(C₆H₅O₇)₂.4H₂O).

6. The method according to claim 5, wherein the precipitating agent is selected from the group consisting of phosphates, carbonates, and sulphates.

7. The method according to claim 4, wherein the preparation comprises a solvent.

8. The method according to claim 7, wherein said solvent comprises an anion.

9. The method according to claim 4, wherein the separation is conducted by centrifugation of the preparation.

10. The method according to claim 4, wherein the MBL preparation is obtained by
preparing a gene expression construct encoding human MBL
peptide or a functional equivalent thereof,
transforming a host cell culture with the construct,
cultivating the host cell culture in a culture medium, thereby obtaining expression and secretion of the polypeptide into the culture medium, and
obtaining a preparation comprising a variety of MBL molecules.

11. The method according to claim 7, wherein said solvent is a culturing medium.

12. The process according to claim 10, wherein the gene expression construct comprises at least one intron sequence from the human MBL gene or a functional equivalent thereof.

13. The process according to claim 10, wherein the gene expression construct comprises at least two exon sequences from the human MBL gene or a functional equivalent thereof.

14. The process according to claim 10, wherein the gene expression construct comprises a cDNA sequence encoding a MBL subunit or a functional equivalent thereof.

15. The process according to claim 10, wherein the host cell culture is cultured in vitro.

16. The process according to claim 10, wherein the host cell culture is an eukaryotic host cell culture.

17. The process according to claim 10, wherein the host cell culture is a mammalian host cell culture.
